(19) United States
(12) Patent Application Publication
(10) Pub. No.: US 2015/0320899 A1
(43) Pub. Date: Nov. 12, 2015

(54) VEHICLE AIR FRESHENING SYSTEM

(71) Applicants: Martin Soliz, Houston, TX (US); Noris Soliz, Houston, TX (US)

(72) Inventors: Martin Soliz, Houston, TX (US); Noris Soliz, Houston, TX (US)

(21) Appl. No.: 14/273,749

(22) Filed: May 9, 2014

(51) Int. Cl. A61L 9/14; F25B 45/00

(52) U.S. Cl. CPC A61L 9/14; F25B 45/00

(57) ABSTRACT

A vehicle air freshening system comprising a canister of Freon; a scent within the canister of Freon; a detachable charging gun attached to a top portion of the canister of Freon; a handle positioned on the detachable charging gun, where the handle activates dispensing the Freon and scent into a vehicle's air conditioner; and a hose attached to the detachable charging gun, where the hose is attached with a connector to the charging gun, and where the hose dispenses the Freon and scent into the vehicle air conditioner. The scent is distributed throughout the vehicle when the air conditioner blows cool air into the cabin.

### BACKGROUND OF THE INVENTION

1. Field of the Invention

The present invention relates to a scented Freon dispenser that injects scented air into the central cooling system of a vehicle.

2. Description of Related Art

Air fresheners are common tools that assist to provide pleasing scents within a building, home or vehicle. At home most people employ sprays, gel forms or plug in oil heaters to distribute fun and fruity scents throughout their house. Further, the air fresheners may be used in locations like bathrooms to mask malodorous aromas commonly associated with using the toilet. The air fresheners are popular because the user can select their favorite scent, and even mix and match the scents according to the room or location where the air freshener is used.

Often people use air fresheners in their cars and trucks to mask odors picked up in the vehicle like food, moisture, cigarettes, stale air, mold and even alcohol. Scented cardboards that hang from a rearview mirror are some of the most common vehicle air fresheners. Likewise, vent clips, oil packets and sprays are also employed. These fresheners are somewhat effective to hide odors but there is typically a lingering odor that remains even after one or more devices are used. Additionally, malodourous aromas are commonly associated with turning on the vehicle as the air conditioner starts. This scent is difficult if not impossible to mask because it has to do with the internal components of the engine not just the cabin.

Therefore, it would be beneficial in the art to provide an air freshener that completely eliminates the odors within a vehicle. It would also be desirable in the art to provide an air freshener that is incorporated into an existing system within the vehicle so that no clutter or mess is present within the vehicle's cabin.

### SUMMARY OF THE INVENTION

In view of the foregoing disadvantages inherent in the prior art, the general purpose of the present invention is to provide a vehicle air freshening system to distribute scented air through a vehicle cabin, configured to include all of the advantages of the prior art, and to overcome the drawbacks inherent therein.

Accordingly, an object of the present invention is to provide a vehicle air freshening system that incorporates a pleasing scent into a canister of Freon that is then distributed into the air conditioner in the vehicle.

Another object of the present invention is to provide a vehicle air freshening system that does not clutter the vehicle cabin by incorporating the scent directly into the air conditioner.

To achieve the above objects, in an aspect of the present invention, a vehicle air freshening system is described comprising a canister of Freon; a scent within the canister of Freon; a detachable charging gun attached to a top portion of the canister of Freon; a handle positioned on the detachable charging gun, where the handle activates dispensing the Freon and scent into a vehicle's air conditioner; and a hose attached to the detachable charging gun, where the hose is attached with a connector to the charging gun, and where the hose dispenses the Freon and scent into the vehicle air conditioner. The scent is distributed throughout the vehicle when the air conditioner blows cool air into the cabin.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawing in which:

FIG. 1 depicts a perspective view of a vehicle air freshening system in accordance with an exemplary embodiment of the present invention.

FIG. 2 depicts a vent with air flow into the vehicle in accordance with the present invention.

### DETAILED DESCRIPTION OF THE DRAWINGS

The present invention relates to a scented Freon dispenser that injects scented air into the central cooling system of a vehicle. The present invention provides a vehicle air freshening system to eliminate bad odors that typically blow out of the air conditioner of a vehicle. The vehicle air freshening system includes a can of Freon for a charging the air conditioner of the vehicle. The Freon can is filled with Freon and a scent. When the air conditioning is charged by the Freon can the scent is also incorporated into the vehicle, so that as the air conditioning blows cold air it also blows the scent. This way the vehicle remains cool and pleasant smelling until the air conditioning needs to be recharged again.

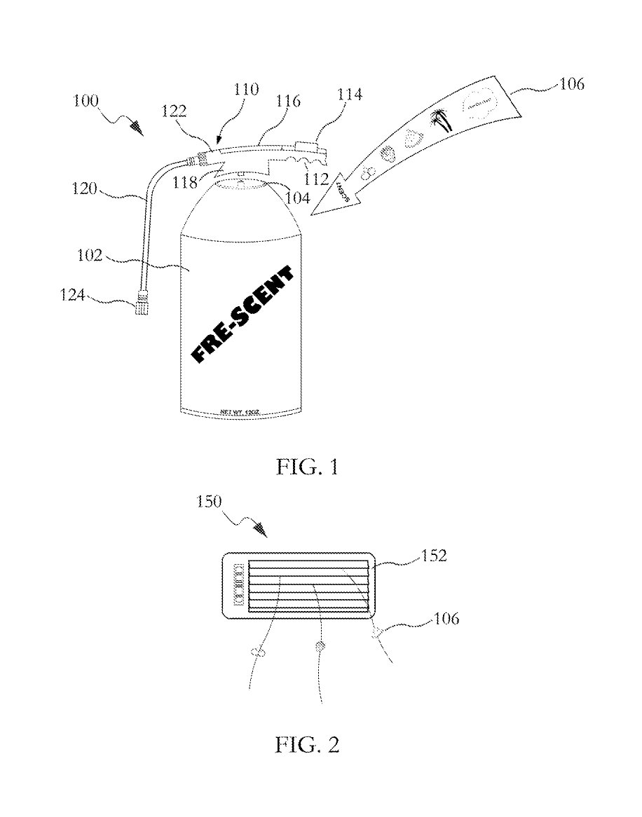

Turning now descriptively to the drawing, referring to FIG. 1, a perspective view of a vehicle air freshening system 100 in accordance with an exemplary embodiment of the present invention. The vehicle air freshening system 100 includes a canister of Freon 102 with a detachable charging gun 110. The canister 102 may be a 12-24 ounce can. The canister of Freon 102 is filled with a scent 106. The scent 106 may be fruits, leather, powders, flowers, tropical scents, clean scents, new car smell, scent blends or other scents that the user finds pleasing.

The charging gun 110 attaches to a top portion 104 of the canister of Freon 102 at a neck 118. The neck 118 may snap on or twist onto the top portion 104 of the canister 102. The neck 118 is positioned below a handle 112. The handle 112 includes an actuator 114 to activate dispensing the internal Freon into a vehicle's air conditioner. The handle 112 further includes a level gauge 116 next to the actuator 114 to indicate how much Freon with scent remains within the system 100.

The charging gun 110 attaches to a hose 120 via a connector 122. The hose 120 facilitates the scent 106 and Freon to enter into the air conditioner. At a far end of the hose 120 is an adapter 124 to connect the hose 120 to a port on the air conditioner. Once connected, the scent 106 and Freon are able to be dispensed into the air conditioner. After charging, the air conditioner pushes a cool scented air 106 into the vehicle through an air vent 150, as shown in FIG. 2. The cool scented air 106 is blown through the slats 152 of the air vent 150 thus providing a lasting air freshener without requiring a scented cardboard or oil dispenser to clutter the vehicle. Each time the air conditioning is activated the scent 106 is distributed throughout the vehicle.

Once the vehicle air freshening system has imparted the Freon and scent into the engine, the bad smells typically associated with the blast of cold air from the air conditioner is eliminated. The scent masks any malodorous aromas that may push through the vents. The vehicle air freshening system is activated as soon as the vehicle is turned on to distribute the pleasing aroma as preferred by the user. The user does not need to use scented hangings, clip-on fresheners or scented packets for smell improvement. The vehicle cabin is left uncluttered by pushing the fresh scent through the vehicle's internal air conditioning system.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A vehicle air freshening system comprising:
a. a canister of Freon;
b. a scent within the canister of Freon;
c. a detachable charging gun attached to a top portion of the canister of Freon;
d. a handle positioned on the detachable charging gun, where the handle activates dispensing the Freon and scent into a vehicle's air conditioner; and
e. a hose attached to the detachable charging gun, where the hose is attached with a connector to the charging gun, and where the hose dispenses the Freon and scent into the vehicle air conditioner.

2. The vehicle air freshening system according to claim 1, where the detachable charging gun includes a level gauge to indicate the amount of Freon and scent that are within the canister.

3. The vehicle air freshening system according to claim 1, where the handle includes an actuator to initiate distributing the Freon and scent into the air conditioner.

4. The vehicle air freshening system according to claim 1, where the canister of Freon is a 12 ounce canister.

5. The vehicle air freshening system according to claim 1, where the canister of Freon is a 24 ounce canister.

6. The vehicle air freshening system according to claim 1, where the scent is one of at least fruit, leather, powders, flowers, new car smell, tropical scents, or scent blends.

7. The vehicle air freshening system according to claim 1, where the hose includes an adapter at an end, where the adapter fastens the hose to a port on the air conditioner and where the adapter facilitates the hose to dispense the Freon and scent into the air conditioner.

8. A method of use for a vehicle air freshening system comprising the step of:
a. attaching a detachable charging gun to a top of a canister filled with Freon and a scent;
b. connecting a hose from the detachable charging gun to a port on an air conditioner within the vehicle;
c. pressing an actuator on a handle of the detachable charging gun to dispense the Freon and scent from the canister into the air conditioner.

9. The method of use for the vehicle air freshening system according to claim 8, further comprising the step of monitoring the amount of Freon in the canister with a gauge on the detachable charging gun.

10. The method of use for the vehicle air freshening system according to claim 8, further comprising the step of distributing the scent throughout the vehicle by turning on the vehicle and activating the air conditioner.
